SEQUENCE LISTING (5 sequences)

SEQ ID NO: 1 - DNA, 2790 bp, human
  CDS (8)...(1273)
cggggacatgaggtggatactgttcattggggcccttattgggtccagcatctgtggccgagaaaaattttttggggaccaagttttgaggattaatgtcagaaatggagacgagatcagcaaattgagtcaactagtgaattcaaacaacttgaagctcaatttctggaaatctccctcctccttcaatcggcctgtggatgtcctggtcccatctgtcagtctgcaggcatttaaatccttcctgagatcccagggcttagagtacgcagtgacaattgaggacctgcaggcccttttagacaatgaagatgatgaaatgcaacacaatgaagggcaagaacggagcagtaataacttcaactacggggcttaccattccctggaagctatttaccacgagatggacaacattgccgcagactttcctgacctggcgaggagggtgaagattggacattcgtttgaaaaccggccgatgtatgtactgaagttcagcactgggaaaggcgtgaggcggccggccgtttggctgaatgcaggcatccattcccgagagtggatctcccaggccactgcaatctggacggcaaggaagattgtatctgattaccagagggatccagctatcacctccatcttggagaaaatggatattttcttgttgcctgtggccaatcctgatggatatgtgtatactcaaactcaaaaccgattatggaggaagacgcggtcccgaaatcctggaagctcctgcattggtgctgacccaaatagaaactggaacgctagttttgcaggaaagggagccagcgacaacccttgctccgaagtgtaccatggaccccacgccaattcggaagtggaggtgaaatcagtggtagatttcatccaaaaacatgggaatttcaagggcttcatcgacctgcacagctactcgcagctgctgatgtatccatatgggtactcagtcaaaaaggccccagatgccgaggaactcgacaaggtggcgaggcttgcggccaaagctctggcttctgtgtcgggcactgagtaccaagtgggtcccacctgcaccactgtctatccagctagcgggagcagcatcgactgggcgtatgacaacggcatcaaatttgcattcacatttgagttgagagataccgggacctatggcttcctcctgccagctaaccagatcatccccactgcagaggagacgtggctggggctgaagaccatcatggagcatgtgcgggacaacctctactaggcgatggctctgctctgtctacatttatttgtacccacacgtgcacgcactgaggccattgttaaaggagctctttcctacctgtgtgagtcagagccctctgggtttgtggagcacacaggcctgcccctctccagccagctccctggagtcgtgtgtcctggcggtgtccctgcaagaactggttctgccagcctgctcaattttggtcctgctgtttttgatgagccttttgtctgtttctccttccaccctgctggctgggcggctgcactcagcatcaccccttcctgggtggcatgtctctctctacctcatttttagaaccaaagaacatctgagatgattctctaccctcattcacatctagccaagccagtgacctttgctctggtggcactgtgggagacaccacttgtctttaggtgggtctcaaagatgatgtagaatttcctttaatttctcgcagtcttcctggaaaatattttcctttgagcagcaaatcttgtagggatatcagtgaaggtctctccctccctcctctcctgttttttttttttgaggcagagttttgctcttgttgcccaggctggagtgtgatgggctcgatcttggctcaccacaacctctgcctcctgggttcaagcaattctcctgcctcagcctcttgagtagcttggtttataggcgcatgccaccatgcctggctaattttgtgtttttagtagagacagggtttctccatgttggtcaggctggtctcaaactcccaacctcaggtgatctgccctccttggcctcccagagtgctgggattacagggggagccactgtgccggtcccgtcccctccttttttaggcctgaatacaaagtagaagatcactttccttcactgtgctgagaatttctagatactacagttcttactcctctcttccctttgttattcagtgtgaccaggatgggcgggaggggatctgtgtcactgtaggtactgtgcccaggaaggctgggtgaagtccccatctaaattgcaggatggcgaaattatccccatctgtcctaatgggcttccctcctctttgccttttgaactcacttcaaagatgtaggcctcatcttacaggtcctaaatcactcatctggcctggataatctcactgccctggcacattcccatttgtgctggggtatcctgtgtttccttgtcctggtttgtgtgtgtgtgtgtgtgtgtgtgtgtgtgtgtgtgtgtttgtgtgtgtgtgtctgtctattttgatccggcccaagttcctaagtagagcaagaattcatcaaccagctgcctttgtttcatttcacctcagcacgtaccatcgtcctttggggggttgtttgtttttgttttttgctttaaccaaaatgtttgtaaatcttaacctcctgcctaggatttgtacagcatttggtgtgtgcttataagccaataaatattcaatgtgagttccaaaaaaaaaaaaaaaaaaaaaaaaa

SEQ ID NO: 2 - protein, 421 aa, human
MRWILFIGALIGSSICGREKFFGDQVLRINVRNGDEISKLSQLVNSNNLKLNFWKSPSSFNRPVDVLVPSVSLQAFKSFLRSQGLEYAVTIEDLQALLDNEDDEMQHNEGQERSSNNFNYGAYHSLEAIYHEMDNIAADFPDLARRVKIGHSFENRPMYVLKFSTGKGVRRPAVWLNAGIHSREWISQATAIWTARKIVSDYQRDPAITSILEKMDIFLLPVANPDGYVYTQTQNRLWRKTRSRNPGSSCIGADPNRNWNASFAGKGASDNPCSEVYHGPHANSEVEVKSVVDFIQKHGNFKGFIDLHSYSQLLMYPYGYSVKKAPDAEELDKVARLAAKALASVSGTEYQVGPTCTTVYPASGSSIDWAYDNGIKFAFTFELRDTGTYGFLLPANQIIPTAEETWLGLKTIMEHVRDNLY

SEQ ID NO: 3 - DNA, 1266 bp, human
  CDS (1)...(1266)
atgaggtggatactgttcattggggcccttattgggtccagcatctgtggccgagaaaaattttttggggaccaagttttgaggattaatgtcagaaatggagacgagatcagcaaattgagtcaactagtgaattcaaacaacttgaagctcaatttctggaaatctccctcctccttcaatcggcctgtggatgtcctggtcccatctgtcagtctgcaggcatttaaatccttcctgagatcccagggcttagagtacgcagtgacaattgaggacctgcaggcccttttagacaatgaagatgatgaaatgcaacacaatgaagggcaagaacggagcagtaataacttcaactacggggcttaccattccctggaagctatttaccacgagatggacaacattgccgcagactttcctgacctggcgaggagggtgaagattggacattcgtttgaaaaccggccgatgtatgtactgaagttcagcactgggaaaggcgtgaggcggccggccgtttggctgaatgcaggcatccattcccgagagtggatctcccaggccactgcaatctggacggcaaggaagattgtatctgattaccagagggatccagctatcacctccatcttggagaaaatggatattttcttgttgcctgtggccaatcctgatggatatgtgtatactcaaactcaaaaccgattatggaggaagacgcggtcccgaaatcctggaagctcctgcattggtgctgacccaaatagaaactggaacgctagttttgcaggaaagggagccagcgacaacccttgctccgaagtgtaccatggaccccacgccaattcggaagtggaggtgaaatcagtggtagatttcatccaaaaacatgggaatttcaagggcttcatcgacctgcacagctactcgcagctgctgatgtatccatatgggtactcagtcaaaaaggccccagatgccgaggaactcgacaaggtggcgaggcttgcggccaaagctctggcttctgtgtcgggcactgagtaccaagtgggtcccacctgcaccactgtctatccagctagcgggagcagcatcgactgggcgtatgacaacggcatcaaatttgcattcacatttgagttgagagataccgggacctatggcttcctcctgccagctaaccagatcatccccactgcagaggagacgtggctggggctgaagaccatcatggagcatgtgcgggacaacctctactag

SEQ ID NO: 4 - DNA, 388 bp, murine
  CDS (0)...(0)
cgcgtccgtttgcaggagaggggaccggtgataacccttgctctgaagtgtaccatggctcccaccccaattctgaagtggaggtgaaatcggtggtggatttcattcaaaagcatgggaacttcaaatgcttcattgacctgcatagctactcacagctgctgatgtatccctatgggtacacagtcaagaaggccccggatgctgaggagctggacgatgtggcgaggaatgcagcccaagccctggcttctctctcgggcactaagtaccgagtgggcccaacttgcaccaccgtctatccagctagtgggagcagcgttgactgggcatacgacaatggcatcaagtatgctttcacatttgagctgagagacactgggtacta

SEQ ID NO: 5 - protein, 125 aa, murine
FAGEGTGDNPCSEVYHGSHPNSEVEVKSVVDFIQKHGNFKCFIDLHSYSQLLMYPYGYTVKKAPDAEELDDVARNAAQALASLSGTKYRVGPTCTTVYPASGSSVDWAYDNGIKYAFTFELRDTG

(19) United States
(12) Patent Application Publication
(10) Pub. No.: US 2004/0077001 A1
(43) Pub. Date: Apr. 22, 2004

(54) Use for carboxypeptidase-A4 in the diagnosis and treatment of metabolic disorders

(75) Inventor: David White, Braintree, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals Inc.

(21) Appl. No.: 10/631,702

(22) Filed: Jul. 31, 2003

Related U.S. Application Data
(60) Provisional application No. 60/400,832, filed on Aug. 2, 2002.

(51) Int. Cl. C12Q 1/68

(52) U.S. Cl. 435/6

(57) ABSTRACT

The present invention relates to methods and compositions for the diagnosis and treatment of metabolic disorders, including, but not limited to, obesity, diabetes, overweight anorexia, or cachexia. The present invention describes methods for the diagnostic evaluation and prognosis of various metabolic disorders and obesity, for the identification of subjects exhibiting a predisposition to such conditions, as well as the diagnostic monitoring of patients undergoing clinical evaluation for the treatment of metabolic disease and obesity, and for monitoring the efficacy of compounds in clinical trials. The invention further provides methods for identifying a compound capable of modulating a metabolic activity as well as treating a metabolic disorder. In addition, the invention provides methods useful for modulating a metabolic activity as well as for treating a subject having a metabolic disorder characterized by either aberrant CPA4 polypeptide activity or aberrant CPA4 nucleic acid expression.

### CROSS-REFERENCE TO RELATED APPLICATIONS

This application claims the benefit of U.S. Provisional Application No. 60/400,832 filed Aug. 2, 2002, the contents of which are incorporated herein by this reference.

### BACKGROUND OF THE INVENTION

Obesity represents the most prevalent of body weight disorders, affecting an estimated 30 to 50% of the middle-aged population in the western world. Other body weight disorders, such as anorexia nervosa and bulimia nervosa, which together affect approximately 0.2% of the female population of the western world, also pose serious health threats. Further, such disorders as anorexia and cachexia (wasting) are also prominent features of other diseases such as cancer, cystic fibrosis, and AIDS.

Obesity, defined as a body mass index (BMI) of 30 kg/²m or more, contributes to diseases such as coronary artery disease, hypertension, stroke, diabetes, hyperlipidemia and some cancers. (See, e.g., Nishina, P. M. et al. (1994), Metab. 43:554-558; Grundy, S. M. & Barnett, J. P. (1990), Dis. Mon. 36:641-731). Obesity is a complex multifactorial chronic disease that develops from an interaction of genotype and the environment and involves social, behavioral, cultural, physiological, metabolic and genetic factors.

Diabetes mellitus is the most common metabolic disease worldwide. Daily 1700 new cases of diabetes are diagnosed in the United States, while at least one-third of the 16 million Americans with diabetes are unaware of it. Diabetes is the leading cause of blindness, renal failure, and lower limb amputations in adults and is a major risk factor for cardiovascular disease and stroke. Normal glucose homeostasis requires the finely tuned orchestration of insulin secretion by pancreatic beta cells in response to subtle changes in blood glucose levels, delicately balanced with secretion of counter-regulatory hormones such as glucagon. One of the fundamental actions of insulin is to stimulate uptake of glucose from the blood into tissues, especially muscle and fat. Type 1 diabetes results from autoimmune destruction of pancreatic beta cells causing insulin deficiency. Type 2 or non-insulin-dependent diabetes mellitus (NIDDM) accounts for >90% of cases and is characterized by a triad of (1) resistance to insulin action on glucose uptake in peripheral tissues, especially skeletal muscle and adipocytes, (2) impaired insulin action to inhibit hepatic glucose production, and (3) misregulated insulin secretion (DeFronzo, (1997) Diabetes Rev. 5:177-269). In most cases, type 2 diabetes is a polygenic disease with complex inheritance patterns (reviewed in Kahn et al., (1996) Annu. Rev. Med. 47:509-531).

The identification of the role of insulin in the control of body weight was the initial identification of a hormonal signal implicated in the hormonal regulation of metabolism via the central nervous system. Subsequently, leptin, a hormone secreted by adipocytes, was identified as an additional adiposity signal. Both insulin and leptin have been shown to circulate at levels proportional to body fat content; receptors for each of these molecules have been identified in neurons in the hypothalamus, a homeostasis control center in the brain; and increases of either hormone injected into the brain result in reduced food intake while deficiency has the opposing effect (reviewed in Schwartz et al., (2000) Nature 404:661-671).

In addition to hormonal controls, neurotransmitters (e.g., serotonin and norepinephrine (NE)) as well as neuropeptides (e.g., NPY, MCH, galanin, beta-endorphin, and dynorphin) are also known to affect feeding behavior and have been implicated in regulating food intake. For example, melanin-concentrating hormone (MCH) is a cyclic peptide implicated in playing a role in regulation of feeding because of localization and biological activities of mammalian MCH peptide. The gene encodes an MCH peptide, as well as, a 13 amino acid peptide which is processed and released by hypothalamic cells in culture (Parkes et al (1992) Endocrinology 131:1826-1831). In mammals, MCH gene expression is localized to the lateral hypothalamus, MCH perikarya project throughout the mammalian brain, and it is likely that MCH is involved in integrative processes which accompany complex behaviors (Breton et al. (1993) Molecular and Cellular Neurosciences 4:271-284; Skofitch et al. (1985) Brain Res. Bull. 15:635-639; Zhang et al. (1994) Nature 372:425-432). Two potent orexigenic agents orexin A and B have also been shown to have very similar localization to MCH in the lateral hypothalamus (Sakurai et al., 1998).

Together these data suggest a role for endogenous neuropeptides in the regulation of energy balance and response to stress, and provide a rationale for the development of specific compounds acting via modulation of production and activity of neuropeptides for use in the treatment of metabolic disorders, including obesity and stress-related disorders.

### SUMMARY OF THE INVENTION

The present invention provides methods and compositions for the diagnosis and treatment of metabolic disorders, e.g., obesity, anorexia, cachexia, and diabetes. The present invention is based, at least in part, on the discovery that carboxypeptidase A4 (CPA4) molecules are expressed at high levels in hypothalamic tissue. CPA4 nucleic acid and polypeptide molecules may play a role in or function in regulation of maturation of neuropeptides involved in metabolic signaling pathways. In one embodiment, the CPA4 molecules modulate the activity of one or more peptides involved in an orexigenic signaling pathway, e.g., a neuropeptide signaling pathway involved in regulation of satiety controls and functioning.

Accordingly, the present invention provides methods for the diagnosis and treatment of metabolic disorders including but not limited to obesity, anorexia, cachexia, and diabetes.

In one aspect, the invention provides methods for identifying a nucleic acid or a polypeptide associated with a metabolic disorder, e.g., obesity, anorexia, cachexia, and diabetes. Methods include contacting a sample expressing a CPA4 nucleic acid or polypeptide with a test compound and assaying the ability of the test compound to modulate the expression of a CPA4 nucleic acid or the activity of a CPA4 polypeptide.

In another aspect, the invention provides methods for identifying a compound capable of treating a metabolic disorder, e.g., obesity, anorexia, cachexia, and diabetes. Methods include assaying the ability of the compound to modulate CPA4 nucleic acid expression or CPA4 polypeptide activity. In one embodiment, the ability of the compound to modulate nucleic acid expression or CPA4 polypeptide activity is determined by detecting carboxypeptidase activity. In another embodiment, the ability of the compound to modulate nucleic acid expression or CPA4 polypeptide activity is determined by detecting modulation of satiety signals, modulation of feeding behavior, or modulation of metabolic functioning.

In another aspect, the invention provides methods for identifying a compound capable of modulating a hypothalamic neuropeptide activity, e.g., satiety controls, feeding activity, or thermogenesis. The method includes contacting a cell capable of expressing a CPA4 nucleic acid or polypeptide with a test compound and assaying the ability of the test compound to modulate the expression of a CPA4 nucleic acid or the activity of a CPA4 polypeptide, such that modulation of CPA4 nucleic acid expression or activity of CPA4 results in modified neuropeptide signaling activity.

In another aspect, the invention provides methods for modulating a hypothalamic neuropeptide signaling activity, e.g., satiety controls, feeding activity, or thermogenesis. The method includes contacting a cell with a CPA4 modulator, for example, an anti-CPA4 antibody, a CPA4 polypeptide comprising the amino acid sequence of SEQ ID NO:2 or a fragment thereof, a CPA4 polypeptide comprising an amino acid sequence which is at least 90 percent identical to the amino acid sequence of SEQ ID NO:2, an isolated naturally occurring allelic variant of a polypeptide consisting of the amino acid sequence of SEQ ID NO:2, a small molecule, a CPA4 substrate, a CPA4 substrate peptidomimetic, an anti-sense CPA4 nucleic acid molecule, a nucleic acid molecule of SEQ ID NO:1 or a fragment thereof, or a ribozyme.

In yet another aspect, the invention features a method for identifying a subject having a metabolic disorder characterized by aberrant CPA4 polypeptide activity or aberrant CPA4 nucleic acid expression, e.g., obesity, anorexia, or cachexia. The method includes contacting a sample obtained from the subject and expressing a CPA4 nucleic acid or polypeptide with a test compound and assaying the ability of the test compound to modulate the expression of a CPA4 nucleic acid or the activity of a CPA4 polypeptide.

In yet another aspect, the invention features a method for treating a subject having a metabolic disorder characterized by aberrant CPA4 polypeptide activity or aberrant CPA4 nucleic acid expression, e.g., obesity, diabetes, anorexia, or cachexia. The method includes administering to the subject a CPA4 modulator, e.g., in a pharmaceutically acceptable formulation or by using a gene therapy vector. Embodiments of this aspect of the invention include the CPA4 modulator being a small molecule, an anti-CPA4 antibody, a CPA4 polypeptide comprising the amino acid sequence of SEQ ID NO:2 or a fragment thereof, a CPA4 polypeptide comprising an amino acid sequence which is at least 90 percent identical to the amino acid sequence of SEQ ID NO:2, an isolated naturally occurring allelic variant of a polypeptide consisting of the amino acid sequence of SEQ ID NO:2, a CPA4 substrate, a CPA4 substrate peptidomimetic, an antisense CPA4 nucleic acid molecule, a nucleic acid molecule of SEQ ID NO:1 or a fragment thereof, or a ribozyme.

Other features and advantages of the invention will be apparent from the following detailed description and claims.

### DETAILED DESCRIPTION OF THE INVENTION

The invention provides methods and compositions for the diagnosis and treatment of metabolic disorders, e.g., obesity, diabetes, anorexia, and cachexia. The present invention is based, at least in part, on the discovery that the carboxypeptidase A4 (CPA4) nucleic acid and polypeptide molecules are expressed at high levels in brain tissue, particularly localized to the hypothalamus. The murine CPA4 transcript is enriched in the arcuate/paraventricular nuclei of the hypothalamus; over-expressed in the arcuate nucleus of ob/ob mice; and induced in the arcuate nucleus in response to overnight fasting conditions. The pattern of expression and regulation of CPA4 is suggestive of a role in regulating the maturation of orexigenic peptides in the body weight nuclei of the hypothalamus. Without intending to be limited by mechanism, it is believed that CPA4 molecules can modulate body weight homeostasis by (directly or indirectly) affecting the rate of metabolism and/or satiety controls.

Carboxypeptidase A3 (Genbank accession no.: AF095719) was identified as a gene induced by histone deacetylase inhibitors during differentiation in prostate cancer cell. See, e.g., Huang, H. et al., (1999) Cancer Research 59: 2981-2988, which is incorporated herein by reference. The gene encodes a protein which shares highest homology to metallocarboxypeptidase family member CPA2 (63% and 61% to human and rat, respectively). Additionally, the encoded protein shares high homology to CPA1. In particular, critical residues believed to be involved in active sites and metal binding regions (e.g., Zn+2 binding and CP domain), are highly conserved throughout the family members. See, Huang, H. et al., (1999) Cancer Research 59: 2981-2988, which is incorporated herein by reference.

Although the genbank sequence AF095719 was originally identified as CPA3 in the literature, it was subsequently renamed CPA4 by the human genome nomenclature committee (Wei, S., et al., (2002) J. Biol. Chem. 277: 14954-14964). The sequence is thus termed CPA4 throughout this specification. The nucleotide sequence of human CPA4, also referred to herein as 17683, is depicted in SEQ ID NO: 1. The amino acid sequence corresponds to amino acids 1 to 421 of SEQ ID NO: 2. The coding region of human CPA4 without the 5′ and 3′ untranslated regions of the human CPA4 gene is shown in SEQ ID NO: 3.

A partial murine CPA4 sequence has been identified. The nucleotide sequence m17683 or mCPA4 is depicted in SEQ ID NO:4. The partial amino acid sequence of mCPA4 is depicted in SEQ ID NO:5.

As used herein, the term “metabolic disorder” includes a disorder, disease or condition which is caused or characterized by an abnormal metabolism (i.e., the chemical changes in living cells by which energy is provided for vital processes and activities) in a subject. Metabolic disorders include diseases, disorders, or conditions associated with aberrant feeding activity or aberrant neuronal (e.g., hypothalamic neuronal cell) signaling or function. Metabolic disorders can be characterized by a misregulation (e.g., downregulation or upregulation) of CPA4 activity. Metabolic disorders can detrimentally affect cellular functions such as cellular proliferation, growth, differentiation, or migration, cellular regulation of homeostasis, inter- or intra-cellular communication; tissue function, such as liver function, muscle function, or adipocyte function; systemic responses in an organism, such as hormonal responses (e.g., insulin and/or leptin response) or satiety responses. Examples of metabolic disorders include obesity, diabetes, hyperphagia, endocrine abnormalities, triglyceride storage disease, Bardet-Biedl syndrome, Lawrence-Moon syndrome, Prader-Labhart-Willi syndrome, hypophagia, anorexia, and cachexia. Obesity is defined as a body mass index (BMI) of 30 kg/²m or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/m or more, 26 kg/²m or more, 27 kg/²m or more, 28 kg/²m or more, 29 kg/²m or more, 29.5 kg/²m or more, or 29.9 kg/²m or more, all of which are typically referred to as overweight (National Institute of Health, Clinical Guidelines on the Identification, Evaluation, and Treatment of Overweight and Obesity in Adults (1998)).

As used interchangeably herein, “CPA4 activity,” “biological activity of CPA4” or “functional activity of CPA4,” includes an activity exerted by a CPA4 protein, polypeptide or nucleic acid molecule on a CPA4 responsive cell or tissue, e.g., hypothalamic neurons, or on a CPA4 protein substrate, as determined in vivo, or in vitro, according to standard techniques. CPA4 activity can be a direct activity, such as an association with a CPA4-target molecule. As used herein, a “ligand” or “substrate” or “target molecule” or “binding partner” is a molecule with which a CPA4 protein binds or interacts in nature, such that CPA4-mediated function, e.g., modulation of metabolism, is achieved. A CPA4 target molecule can be a non-CPA4 molecule or a CPA4 protein or polypeptide. Examples of such target molecules include proteins in the same signaling path as the CPA4 protein, e.g., proteins which may function upstream (including both stimulators and inhibitors of activity) or downstream of the CPA4 protein in a pathway involving regulation of metabolism. CPA4 target molecules may include for example small molecules, and peptidomimetics. Alternatively, a CPA4 activity is an indirect activity, such as a cellular signaling activity mediated by interaction of the CPA4 protein with a CPA4 target molecule. The biological activities of CPA4 are described herein. For example, the CPA4 proteins can have one or more of the following activities: 1) modulation of fat homeostasis; 2) modulation of production neuropeptide production; 3) modulation of hypothalamic neuropeptide signaling activity; 4) modulation of satiety controls in the brain; 5) modulation of energy expenditure 6) modulation of feeding behavior 7) modulation of signaling for maintenance of energy homeostasis, 8) interaction with (e.g., bind to) peptide substrates; 12) modulation of the release of a neurotransmitter, e.g., acetylcholine, from a neuron, e.g., a presynaptic neuron; 13) modulation of an acetylcholine response in an acetylcholine responsive cell (e.g., a neuronal cell) to, for example, beneficially affect the acetylcholine responsive cell, e.g., a neuron; 14) signaling of substrate binding via phosphatidylinositol turnover; and 15) modulation of, e.g., activation or inhibition, phospholipase C activity.

As used herein, “metabolic activity” may include an activity exerted by a cell, e.g., a neuronal cell such as for example a hypothalamic neuronal cell, or an activity that takes place in a neuronal cell. For example, such activities include cellular processes that contribute to the physiological role of hypothalamic neuronal cells (whether directly or indirectly, e.g., through signaling), in regulation of metabolism and satiety controls and include, but are not limited to, cell proliferation, differentiation, growth, migration, programmed cell death, uncoupled mitochondrial respiration, thermogenesis, and transmission of neurotransmitters.

Various aspects of the invention are described in further detail in the following subsections:

Screening Assays:

The invention provides methods (also referred to herein as a “screening assays”) for identifying modulators, i.e., candidate or test compounds or agents (e.g., peptides, peptidomimetics, small molecules or other drugs) which bind to CPA4 polypeptides, have a stimulatory or inhibitory effect on, for example, CPA4 expression or CPA4 activity, or have a stimulatory or inhibitory effect on, for example, the expression or activity of CPA4 substrate.

In one embodiment, the invention provides assays for screening candidate or test compounds which are substrates of a CPA4 protein or polypeptide or biologically active portion thereof. In another embodiment, the invention provides assays for screening candidate or test compounds which bind to or modulate the activity of a CPA4 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

Expression of CPA4 or activity of CPA4 can be determined by, for example, a carboxipeptidase assay. Briefly, 5 μl of serum of mice, for example, can be combined with 45 μl of 55 ΞM of an appropriate 17865 substrate including but not limited to e.g., angiotensin I, a kinin, or kinetensin, in an appropriate buffer. Then, the rate of proteolytic degradation of the substrate can be measured by measuring the production of fluorescence (in flurorescence units) per second for 30 minutes at room temperature at a gain setting of 10. The average rate of fluoresence units per second (FU/sec) correlates directly with the amount of CPA4 in the serum. As a control for the specificity of CPA4, a standard carboxypeptidase assay can be performed (Holmquist and Riordan, Carboxypeptidase A, pp 44-60, Peptidase and their Inhibitors in Method of Enzymatic Analysis (1984)). Further, an additional carboxypeptidase assay can be performed in accordance with that described in Ostrowska, H. et al. (1998) Rocz Akad. Med. Bialymst., 43:39-55, which is incorporated herein by reference.

In one embodiment, an assay is a cell-based assay in which a cell which expresses a CPA4 polypeptide or biologically active portion thereof is contacted with a test compound and the ability of the test compound to modulate CPA4 activity is determined. Determining the ability of the test compound to modulate CPA4 activity can be accomplished by monitoring, for example, neuropeptide production, neuropeptide signaling, phosphatidylinositol turnover, phospholipase C activity, or carboxypeptidase activity in a cell, or neurotransmitter secretion from a cell. The cell, can be of mammalian origin, including human, non-human, or primate origin for example. The cell may be a recombinant cell or a cell which naturally expresses CPA4, e.g., a nerve cell.

The ability of the test compound to modulate CPA4 binding to a substrate or to bind to CPA4 can also be determined. Determining the ability of the test compound to modulate CPA4 binding to a substrate can be accomplished, for example, by coupling a CPA4 substrate with a radioisotope, an enzymatic label, or a fluorescent label such that binding of the CPA4 substrate to CPA4 can be determined by detecting the labeled CPA4 substrate in a complex. Alternatively, CPA4 could be coupled with a radioisotope, enzymatic, or fluorescent label to monitor the ability of a test compound to modulate CPA4 binding to a CPA4 substrate in a complex. Determining the ability of the test compound to bind CPA4 can be accomplished, for example, by coupling the compound with a radioisotope, enzymatic, or fluorescent label such that binding of the compound to CPA4 can be determined by detecting the labeled CPA4 compound in a complex. For example, compounds (e.g., CPA4 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product. Compounds can be fluorescently labeled with, for example, fluorescein, rhodamine, AMCA, or TRF, and the fluorescent label detected by exposure of the compound to a specific wavelength of light.

It is also within the scope of this invention to determine the ability of a compound (e.g., a CPA4 substrate) to interact with CPA4 without the labeling of any of the interactants. For example, a microphysiometer can be used to detect the interaction of a compound with CPA4 without the labeling of either the compound or the CPA4. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and CPA4.

In another embodiment, an assay is a cell-based assay comprising contacting a cell expressing a CPA4 target molecule (e.g., a CPA4 substrate) with a test compound and determining the ability of the test compound to modulate (e.g., stimulate or inhibit) the activity of the CPA4 target molecule. Determining the ability of the test compound to modulate the activity of a CPA4 target molecule can be accomplished, for example, by determining the ability of the CPA4 polypeptide to bind to or interact with the CPA4 target molecule.

Determining the ability of the CPA4 polypeptide, or a biologically active fragment thereof, to bind to or interact with a CPA4 target molecule can be accomplished by one of the methods described above for determining direct binding. In a preferred embodiment, determining the ability of the CPA4 polypeptide to bind to or interact with a CPA4 target molecule can be accomplished by determining the activity of the target molecule. For example, the activity of the target molecule can be determined by detecting 2+induction of a cellular second messenger of the target (i.e., intra-cellular Ca diacylglycerol, IP₃, and the like), detecting signaling activity of the target using an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which a CPA4 polypeptide or biologically active portion thereof is contacted with a test compound and the ability of the test compound to bind to the CPA4 polypeptide or biologically active portion thereof is determined. Preferred biologically active portions of the CPA4 polypeptides to be used in assays of the present invention include fragments which participate in interactions with non-CPA4 molecules, e.g., fragments with high surface probability scores. Binding of the test compound to the CPA4 polypeptide can be determined either directly or indirectly as described above. In a preferred embodiment, the assay includes contacting the CPA4 polypeptide or biologically active portion thereof with a known compound which binds CPA4 to form an assay mixture, contacting the assay mixture with a test compound, and determining the ability of the test compound to interact with a CPA4 polypeptide, wherein determining the ability of the test compound to interact with a CPA4 polypeptide comprises determining the ability of the test compound to preferentially bind to CPA4 or biologically active portion thereof as compared to the known compound.

In another embodiment, the assay is a cell-free assay in which a CPA4 polypeptide or biologically active portion thereof is contacted with a test compound and the ability of the test compound to modulate (e.g., stimulate or inhibit) the activity of the CPA4 polypeptide or biologically active portion thereof is determined. Determining the ability of the test compound to modulate the activity of a CPA4 polypeptide can be accomplished, for example, by determining the ability of the CPA4 polypeptide to bind to a CPA4 target molecule by one of the methods described above for determining direct binding. Determining the ability of the CPA4 polypeptide to bind to a CPA4 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a CPA4 polypeptide can be accomplished by determining the ability of the CPA4 polypeptide to further modulate the activity of a downstream effector of a CPA4 target molecule (e.g., MCH for example). For example, the activity of the effector molecule on an appropriate target can be determined or the binding of the effector to an appropriate target can be determined as previously described.

In yet another embodiment, the cell-free assay involves contacting a CPA4 polypeptide or biologically active portion thereof with a known compound which binds the CPA4 polypeptide to form an assay mixture, contacting the assay mixture with a test compound, and determining the ability of the test compound to interact with the CPA4 polypeptide, wherein determining the ability of the test compound to interact with the CPA4 polypeptide comprises determining the ability of the CPA4 polypeptide to preferentially bind to or modulate the activity of a CPA4 target molecule.

In more than one embodiment of the above assay methods of the present invention, it may be desirable to immobilize either CPA4 or its target molecule to facilitate separation of complexed from uncomplexed forms of one or both of the proteins, as well as to accommodate automation of the assay. Binding of a test compound to a CPA4 polypeptide, or interaction of a CPA4 polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/CPA4 fusion proteins or glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or CPA4 polypeptide, and the mixture incubated under conditions conducive to complex formation (e.g., at physiological conditions for salt and pH). Following incubation, the beads or micrometer plate wells are washed to remove any unbound components, the matrix immobilized in the case of beads, complex determined either directly or indirectly, for example, as described above. Alternatively, the complexes can be dissociated from the matrix, and the level of CPA4 binding or activity determined using standard techniques.

Other techniques for immobilizing proteins on matrices can also be used in the screening assays of the invention. For example, either a CPA4 polypeptide or a CPA4 target molecule can be immobilized utilizing conjugation of biotin and streptavidin. Biotinylated CPA4 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with CPA4 polypeptide or target molecules but which do not interfere with binding of the CPA4 polypeptide to its target molecule can be derivatized to the wells of the plate, and unbound target or CPA4 polypeptide trapped in the wells by antibody conjugation. Methods for detecting such complexes, in addition to those described above for the GST-immobilized complexes, include immunodetection of complexes using antibodies reactive with the CPA4 polypeptide or target molecule, as well as enzyme-linked assays which rely on detecting an enzymatic activity associated with the CPA4 polypeptide or target molecule.

In another embodiment, modulators of CPA4 expression are identified in a method wherein a cell is contacted with a candidate compound and the expression of CPA4 mRNA or polypeptide in the cell is determined. The level of expression of CPA4 mRNA or polypeptide in the presence of the candidate compound is compared to the level of expression of CPA4 mRNA or polypeptide in the absence of the candidate compound. The candidate compound can then be identified as a modulator of CPA4 expression based on this comparison. For example, when expression of CPA4 mRNA or polypeptide is greater (statistically significantly greater) in the presence of the candidate compound than in its absence, the candidate compound is identified as a stimulator of CPA4 mRNA or polypeptide expression. Alternatively, when expression of CPA4 mRNA or polypeptide is less (statistically significantly less) in the presence of the candidate compound than in its absence, the candidate compound is identified as an inhibitor of CPA4 mRNA or polypeptide expression. The level of CPA4 mRNA or polypeptide expression in the cells can be determined by methods described herein for detecting CPA4 mRNA or polypeptide.

In yet another aspect of the invention, the CPA4 polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with CPA4 (“CPA4-binding proteins” or “CPA4-bp”) and are involved in CPA4 activity. Such CPA4-binding proteins are also likely to be involved in the propagation of signals by the CPA4 polypeptides or CPA4 targets as, for example, downstream elements of a CPA4-mediated signaling pathway. Alternatively, such CPA4-binding proteins are likely to be CPA4 inhibitors.

The two-hybrid system is based on the modular nature of most transcription factors, which consist of separable DNA-binding and activation domains. Briefly, the assay utilizes two different DNA constructs. In one construct, the gene that codes for a CPA4 polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CPA4-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the CPA4 polypeptide.

The ability of a test compound to modulate insulin sensitivity of a cell can be determined by performing an assay in which cells, e.g., adipose cells, are contacted with the test compound, e.g., transformed to express the test compound; incubated with radioactively labeled glucose (¹⁴C glucose); and treated with insulin. An increase or decrease in glucose in the cells containing the test compound as compared to the control cells indicates that the test compound can modulate insulin sensitivity of the cells. Alternatively, the cells containing the test compound can be incubated with a radioactively labeled phosphate source (e.g., [³²P]ATP) and treated with insulin. Phosphorylation of proteins in the insulin pathway, e.g., the insulin receptor, can then be measured. An increase or decrease in phosphorylation of a protein in the insulin pathway in cells containing the test compound as compared to the control cells indicates that the test compound can modulate insulin sensitivity of the cells.

In another aspect, the invention pertains to a combination of two or more of the assays described herein. For example, a modulating agent can be identified using a cellbased or a cell-free assay, and the ability of the agent to modulate the activity of a CPA4 protein can be confirmed in vivo, e.g., in an animal such as an animal model for obesity, diabetes, anorexia, or cachexia. Examples of animals that can be used include the transgenic mouse described in U.S. Pat. No. 5,932,779 that contains a mutation in an endogenous melanocortin-4-receptor (MC4-R) gene; animals having mutations which lead to syndromes that include obesity symptoms (described in, for example, Friedman, J. M. et al. (1991) Mamm. Gen. 1:130-144; Friedman, J. M. and Liebel, R. L. (1992) Cell 69:217-220; Bray, G. A. (1992) Prog. Brain Res. 93:333-341; and Bray, G. A. (1989) Amer. J. Clin. Nutr. 5:891902); the animals described in Stubdal H. et al. (2000) Mol. Cell Biol. 20(3):878-82 (the mouse tubby phenotype characterized by maturity-onset obesity); the animals described in Abadie J. M. et al. Lipids (2000) 35(6):613-20 (the obese Zucker rat (ZR), a genetic model of human youth-onset obesity and type 2 diabetes mellitus); the animals described in Shaughnessy S. et al. (2000) Diabetes 49(6):904-11 (mice null for the adipocyte fatty acid binding protein); the animals described in Loskutoff D. J. et al. (2000) Ann. N.Y. Acad. Sci. 902:272-81 (the fat mouse); or animals having mutations which lead to syndromes that include diabetes (described in, for example, Alleva et al. (2001) J. Clin. Invest. 107:173-180; Arakawa et al. (2001) Br. J. Pharmacol. 132:578-586; Nakamura et al. (2001) Diabetes Res. Clin. Pract. 51:9-20; O'Harte et al. (2001) Regul. Pept. 96:95-104; Yamanouchi et al. (2000) Exp. Anim. 49:259-266; Hoenig et al. (2000) Am. J. Pathol. 157:2143-2150; Reed et al. (2000) Metabolism 49:1390-1394; and Clark et al. (2000) J. Pharmacol. Toxicol. Methods 43: 1-10). Other examples of animals that may be used include non-recombinant, non-genetic animal models of obesity such as, for example, rabbit, mouse, or rat models in which the animal has been exposed to either prolonged cold or long-term over-eating, thereby, inducing hypertrophy and increasing thermogenesis (Himms-Hagen, J. (1990), supra).

This invention further pertains to novel agents identified by the above-described screening assays. Accordingly, it is within the scope of this invention to further use an agent identified as described herein in an appropriate animal model. For example, an agent identified as described herein (e.g., a CPA4 modulating agent, an antisense CPA4 nucleic acid molecule, a CPA4-specific antibody, or a CPA4-binding partner) can be used in an animal model to determine the efficacy, toxicity, or side effects of treatment with such an agent or other molecules derived from such an agent. Alternatively, an agent identified as described herein can be used in an animal model to determine the mechanism of action of such an agent. Furthermore, this invention pertains to uses of novel agents or derivatives thereof identified by the above-described screening assays for treatments as described herein.

Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining CPA4 polypeptide and/or nucleic acid expression as well as CPA4 activity, in the context of a biological sample (e.g., blood, serum, cerebrospinal fluid, cells, tissue) to thereby determine whether an individual is afflicted with a disease or disorder, or is at risk of developing a disorder, associated with aberrant or unwanted CPA4 expression or activity. The invention also provides for prognostic (or predictive) assays for determining whether an individual is at risk of developing a disorder associated with CPA4 polypeptide, nucleic acid expression or activity. For example, mutations in a CPA4 gene can be assayed in a biological sample. Such assays can be used for prognostic or predictive purpose to thereby prophylactically treat an individual prior to the onset of a disorder characterized by or associated with aberrant or unwanted CPA4 polypeptide, nucleic acid expression or activity.

Another aspect of the invention pertains to monitoring the influence of agents (e.g., drugs, compounds) on the expression or activity of CPA4 in clinical trials.

These and other agents are described in further detail in the following sections.

Diagnostic Assays for Metabolic Disorders

An exemplary method for detecting the presence or absence of CPA4 polypeptide or nucleic acid in a biological sample involves obtaining a biological sample from a test subject and contacting the biological sample with a compound or an agent capable of detecting CPA4 polypeptide or nucleic acid (e.g., mRNA, or genomic DNA) that encodes CPA4 polypeptide such that the presence of CPA4 polypeptide or nucleic acid is detected in the biological sample. In another aspect, the present invention provides a method for detecting the presence of CPA4 activity in a biological sample by contacting the biological sample with an agent capable of detecting an indicator of CPA4 activity such that the presence of CPA4 activity is detected in the biological sample. A preferred agent for detecting CPA4 mRNA or genomic DNA is a labeled nucleic acid probe capable of hybridizing to CPA4 mRNA or genomic DNA. The nucleic acid probe can be, for example, the CPA4 nucleic acid set forth in SEQ ID NO:1 or 3, or the DNA sequence of genbank accession no: AF095719, or a portion thereof, such as an oligonucleotide of at least 15, 30, 50, 100, 250 or 500 nucleotides in length and sufficient to specifically hybridize under stringent conditions to CPA4 mRNA or genomic DNA. Other suitable probes for use in the diagnostic assays of the invention are described herein.

A preferred agent for detecting CPA4 polypeptide is an antibody capable of binding to CPA4 polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect CPA4 mRNA, polypeptide, or genomic DNA in a biological sample in vitro as well as in vivo. For example, in vitro techniques for detection of CPA4 mRNA include Northern hybridizations, in situ hybridizations, RT-PCR, and Taqman analyses. In vitro techniques for detection of CPA4 polypeptide include enzyme linked immunosorbent assays (ELISAs), Western blots, immunoprecipitations and immunofluorescence. In vitro techniques for detection of CPA4 genomic DNA include Southern hybridizations. Furthermore, in vivo techniques for detection of CPA4 polypeptide include introducing into a subject a labeled anti-CPA4 antibody. For example, the antibody can be labeled with a radioactive marker whose presence and location in a subject can be detected by standard imaging techniques.

The present invention also provides diagnostic assays for identifying the presence or absence of a genetic alteration characterized by at least one of (i) aberrant modification or mutation of a gene encoding a CPA4 polypeptide; (ii) aberrant expression of a gene encoding a CPA4 polypeptide; (iii) mis-regulation of the gene; and (iii) aberrant post-translational modification of a CPA4 polypeptide, wherein a wild-type form of the gene encodes a polypeptide with a CPA4 activity. “Misexpression or aberrant expression”, as used herein, refers to a non-wild type pattern of gene expression, at the RNA or protein level. It includes, but is not limited to, expression at non-wild type levels (e.g., over or under expression); a pattern of expression that differs from wild type in terms of the time or stage at which the gene is expressed (e.g., increased or decreased expression (as compared with wild type) at a predetermined developmental period or stage); a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene (e.g., a pattern of increased or decreased expression (as compared with wild type) in the presence of an increase or decrease in the strength of the stimulus).

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting CPA4 polypeptide, mRNA, or genomic DNA, such that the presence of CPA4 polypeptide, mRNA or genomic DNA is detected in the biological sample, and comparing the presence of CPA4 polypeptide, mRNA or genomic DNA in the control sample with the presence of CPA4 polypeptide, mRNA or genomic DNA in the test sample.

The invention also encompasses kits for detecting the presence of CPA4 in a biological sample. For example, the kit can comprise a labeled compound or agent capable of detecting CPA4 polypeptide or mRNA in a biological sample; means for determining the amount of CPA4 in the sample; and means for comparing the amount of CPA4 in the sample with a standard. The compound or agent can be packaged in a suitable container. The kit can further comprise instructions for using the kit to detect CPA4 polypeptide or nucleic acid.

Prognostic Assays for Metabolic Disorders

The diagnostic methods described herein can furthermore be utilized to identify subjects having or at risk of developing a disease or disorder associated with aberrant or unwanted CPA4 expression or activity. As used herein, the term “aberrant” includes a CPA4 expression or activity which deviates from the wild type CPA4 expression or activity. Aberrant expression or activity includes increased or decreased expression or activity, as well as expression or activity which does not follow the wild type developmental pattern of expression or the subcellular pattern of expression. For example, aberrant CPA4 expression or activity is intended to include the cases in which a mutation in the CPA4 gene causes the CPA4 gene to be under-expressed or over-expressed and situations in which such mutations result in a non-functional CPA4 polypeptide or a polypeptide which does not function in a wild-type fashion, e.g., a polypeptide which does not interact with a CPA4 substrate, e.g., a carboxypeptidase substrate, or one which interacts with a non-CPA4 substrate, e.g. a non-carboxypeptidase substrate. As used herein, the term “unwanted” includes an unwanted phenomenon involved in a biological response, such as cellular proliferation. For example, the term unwanted includes a CPA4 expression or activity which is undesirable in a subject.

The assays described herein, such as the preceding diagnostic assays or the following assays, can be utilized to identify a subject having or at risk of developing a disorder associated with a misregulation in CPA4 polypeptide activity or nucleic acid expression, such as a fat metabolism disorder. Alternatively, the prognostic assays can be utilized to identify a subject having or at risk for developing a disorder associated with a misregulation in CPA4 polypeptide activity or nucleic acid expression, such as a fat metabolism disorder. Thus, the present invention provides a method for identifying a disease or disorder associated with aberrant or unwanted CPA4 expression or activity in which a test sample is obtained from a subject and CPA4 polypeptide or nucleic acid (e.g., mRNA or genomic DNA) is detected, wherein the presence of CPA4 polypeptide or nucleic acid is diagnostic for a subject having or at risk of developing a disease or disorder associated with aberrant or unwanted CPA4 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted CPA4 expression or activity. For example, such methods can be used to determine whether a subject can be effectively treated with an agent for a metabolism-associated disorder. Thus, the present invention provides methods for determining whether a subject can be effectively treated with an agent for a disorder associated with aberrant or unwanted CPA4 expression or activity in which a test sample is obtained and CPA4 polypeptide or nucleic acid expression or activity is detected (e.g., wherein the abundance of CPA4 polypeptide or nucleic acid expression or activity is diagnostic for a subject that can be administered the agent to treat a disorder associated with aberrant or unwanted CPA4 expression or activity).

The methods of the invention can also be used to detect genetic alterations in a CPA4 gene, thereby determining if a subject with the altered gene is at risk for a disorder characterized by misregulation in CPA4 polypeptide activity or nucleic acid expression, such as a metabolism-associated disorder. In preferred embodiments, the methods include detecting, in a sample of cells from the subject, the presence or absence of a genetic alteration characterized by at least one of an alteration affecting the integrity of a gene encoding a CPA4-polypeptide, or the mis-expression of the CPA4 gene. For example, such genetic alterations can be detected by ascertaining the existence of at least one of 1) a deletion of one or more nucleotides from a CPA4 gene; 2) an addition of one or more nucleotides to a CPA4 gene; 3) a substitution of one or more nucleotides of a CPA4 gene, 4) a chromosomal rearrangement of a CPA4 gene; 5) an alteration in the level of a messenger RNA transcript of a CPA4 gene, 6) aberrant modification of a CPA4 gene, such as of the methylation pattern of the genomic DNA, 7) the presence of a non-wild type splicing pattern of a messenger RNA transcript of a CPA4 gene, 8) a non-wild type level of a CPA4-polypeptide, 9) allelic loss of a CPA4 gene, and 10) inappropriate post-translational modification of a CPA4 polypeptide. As described herein, there are a large number of assays known in the art which can be used for detecting alterations in a CPA4 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the CPA4 gene (see Abravaya et al. (1995) Nucleic Acids Res 0.23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a CPA4 gene under conditions such that hybridization and amplification of the CPA4 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a CPA4 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in CPA4 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in CPA4 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the CPA4 gene and detect mutations by comparing the sequence of the sample CPA4 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the CPA4 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type CPA4 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in CPA4 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a CPA4 sequence, e.g., a wild-type CPA4 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in CPA4 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control CPA4 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnose patients exhibiting symptoms or family history of a metabolic disease or illness involving a CPA4 gene.

Furthermore, any cell type or tissue in which CPA4 is expressed may be utilized in the prognostic assays described herein.

Monitoring of Effects During Clinical Trials

Monitoring the influence of agents (e.g., drugs) on the expression or activity of a CPA4 polypeptide (e.g., the modulation of transport of biological molecules across membranes) can be applied not only in basic drug screening, but also in clinical trials. For example, the effectiveness of an agent determined by a screening assay as described herein to increase CPA4 gene expression, polypeptide levels, or upregulate CPA4 activity, can be monitored in clinical trials of subjects exhibiting decreased CPA4 gene expression, polypeptide levels, or downregulated CPA4 activity. Alternatively, the effectiveness of an agent determined by a screening assay to decrease CPA4 gene expression, polypeptide levels, or downregulate CPA4 activity, can be monitored in clinical trials of subjects exhibiting increased CPA4 gene expression, polypeptide levels, or upregulated CPA4 activity. In such clinical trials, the expression or activity of a CPA4 gene, and preferably, other genes that have been implicated in, for example, a CPA4-associated disorder can be used as a “read out” or markers of the phenotype of a particular cell.

For example, and not by way of limitation, genes, including CPA4, that are modulated in cells by treatment with an agent (e.g., compound, drug or small molecule) which modulates CPA4 activity (e.g., identified in a screening assay as described herein) can be identified. Thus, to study the effect of agents on metabolism-associated disorders, for example, in a clinical trial, cells can be isolated and RNA prepared and analyzed for the levels of expression of CPA4 and other genes implicated in the metabolism-associated disorder, respectively. The levels of gene expression (e.g., a gene expression pattern) can be quantified by northern blot analysis or RT-PCR, as described herein, or alternatively by measuring the amount of polypeptide produced, by one of the methods as described herein, or by measuring the levels of activity of CPA4 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a CPA4 polypeptide, mRNA, or genomic DNA in the preadministration sample; (iii) obtaining one or more post-administration samples from the subject; (iv) detecting the level of expression or activity of the CPA4 polypeptide, mRNA, or genomic DNA in the post-administration samples; (v) comparing the level of expression or activity of the CPA4 polypeptide, mRNA, or genomic DNA in the pre-administration sample with the CPA4 polypeptide, mRNA, or genomic DNA in the post administration sample or samples; and (vi) altering the administration of the agent to the subject accordingly. For example, increased administration of the agent may be desirable to increase the expression or activity of CPA4 to higher levels than detected, i.e., to increase the effectiveness of the agent. Alternatively, decreased administration of the agent may be desirable to decrease expression or activity of CPA4 to lower levels than detected, i.e. to decrease the effectiveness of the agent. According to such an embodiment, CPA4 expression or activity may be used as an indicator of the effectiveness of an agent, even in the absence of an observable phenotypic response.

Electronic Apparatus Readable Media and Arrays

Electronic apparatus readable media comprising CPA4 sequence information is also provided. As used herein, “CPA4 sequence information” refers to any nucleotide and/or amino acid sequence information particular to the CPA4 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” the CPA4 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon CPA4 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the CPA4 sequence information.

A variety of software programs and formats can be used to store the sequence information on the electronic apparatus readable medium. For example, the sequence information can be represented in a word processing text file, formatted in commerciallyavailable software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the CPA4 sequence information.

By providing CPA4 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a CPA4-associated disease or disorder or a pre-disposition to a CPA4-associated disease or disorder, wherein the method comprises the steps of determining CPA4 sequence information associated with the subject and based on the CPA4 sequence information, determining whether the subject has a CPA4 associated disease or disorder or a pre-disposition to a CPA4-associated disease or disorder and/or recommending a particular treatment for the disease, disorder or pre-disease condition.

The present invention further provides in an electronic system and/or in a network, a method for determining whether a subject has a CPA4-associated disease or disorder or a pre-disposition to a disease associated with a CPA4 wherein the method comprises the steps of determining CPA4 sequence information associated with the subject, and based on the CPA4 sequence information, determining whether the subject has a CPA4-associated disease or disorder or a pre-disposition to a CPA4-associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a CPA4-associated disease or disorder or a pre-disposition to a CPA4-associated disease or disorder associated with CPA4, the method comprising the steps of receiving CPA4 sequence information from the subject and/or information related thereto, receiving phenotypic information associated with the subject, acquiring information from the network corresponding to CPA4 and/or a CPA4-associated disease or disorder, and based on one or more of the phenotypic information, the CPA4 information (e.g., sequence information and/or information related thereto), and the acquired information, determining whether the subject has a CPA4-associated disease or disorder or a pre-disposition to a CPA4-associated disease or disorder. The method may further comprise the step of recommending a particular treatment for the disease, disorder or pre-disease condition.

The present invention also provides a business method for determining whether a subject has a CPA4-associated disease or disorder or a pre-disposition to a CPA4-associated disease or disorder, the method comprising the steps of receiving information related to CPA4 (e.g., sequence information and/or information related thereto), receiving phenotypic information associated with the subject, acquiring information from the network related to CPA4 and/or related to a CPA4-associated disease or disorder, and based on one or more of the phenotypic information, the CPA4 information, and the acquired information, determining whether the subject has a CPA4-associated disease or disorder or a pre-disposition to a CPA4-associated disease or disorder. The method may further comprise the step of recommending a particular treatment for the disease, disorder or pre-disease condition.

The invention also includes an array comprising a CPA4 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be CPA4. This allows a profile to be developed showing a battery of genes specifically expressed in one or more tissues.

In addition to such qualitative determination, the invention allows the quantitative measurement of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a CPA4-associated disease or disorder, progression of CPA4-associated disease or disorder, and processes, such a cellular transformation associated with the CPA4-associated disease or disorder.

The array is also useful for ascertaining the effect of the expression of a gene on the expression of other genes in the same cell or in different cells (e.g., ascertaining the effect of CPA4 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including CPA4) that could serve as a molecular target for diagnosis or therapeutic intervention.

Methods of Treatment of Subjects Suffering from Metabolic Disorders:

The present invention provides for both prophylactic and therapeutic methods of treating a subject at risk of (or susceptible to) a disorder or having a disorder associated with aberrant or unwanted CPA4 expression or activity, e.g. a metabolic disorder such as obesity or diabetes. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the CPA4 molecules of the present invention or CPA4 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

A therapeutic agent includes, but is not limited to, small molecules, peptides, antibodies, ribozymes and antisense oligonucleotides.

Prophylactic Methods

In one aspect, the invention provides a method for preventing in a subject, a disease or condition associated with an aberrant or unwanted CPA4 expression or activity, by administering to the subject a CPA4 or an agent which modulates CPA4 expression or at least one CPA4 activity. Subjects at risk for a disease which is caused or contributed to by aberrant or unwanted CPA4 expression or activity can be identified by, for example, any or a combination of diagnostic or prognostic assays as described herein. Administration of a prophylactic agent can occur prior to the manifestation of symptoms characteristic of the CPA4 aberrancy, such that a disease or disorder is prevented or, alternatively, delayed in its progression. Depending on the type of CPA4 aberrancy, for example, a CPA4 molecule, CPA4 agonist or CPA4 antagonist agent can be used for treating the subject. The appropriate agent can be determined based on screening assays described herein.

Therapeutic Methods

The CPA4 nucleic acid molecules, fragments of CPA4 polypeptides, and anti-CPA4 antibodies (also referred to herein as “active compounds”) of the invention can be incorporated into pharmaceutical compositions suitable for administration. Such compositions typically comprise the nucleic acid molecule, polypeptide, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a CPA4 polypeptide or an anti-CPA4 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a polypeptide or antibody can include a single treatment or, preferably, can include a series of treatments.

In a preferred example, a subject is treated with antibody or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Pharmacogenomics

The CPA4 molecules of the present invention, as well as agents, or modulators which have a stimulatory or inhibitory effect on CPA4 activity (e.g., CPA4 gene expression) as identified by a screening assay described herein can be administered to individuals to treat (prophylactically or therapeutically) metabolism-associated disorders (e.g., feeding disorders) associated with aberrant or unwanted CPA4 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a CPA4 molecule or CPA4 modulator as well as tailoring the dosage and/or therapeutic regimen of treatment with a CPA4 molecule or CPA4 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/E drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a CPA4 polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a CPA4 molecule or CPA4 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a CPA4 molecule or CPA4 modulator, such as a modulator identified by one of the exemplary screening assays described herein.

Recombinant Expression Vectors and Host Cells Used in the Methods of the Invention

The methods of the invention (e.g., the screening assays described herein) include the use of vectors, preferably expression vectors, containing a nucleic acid encoding a CPA4 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., CPA4 proteins, mutant forms of CPA4 proteins, fusion proteins, and the like).

The recombinant expression vectors to be used in the methods of the invention can be designed for expression of CPA4 proteins in prokaryotic or eukaryotic cells. For example, CPA4 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in CPA4 activity assays, (e.g., direct assays or competitive assays described in detail below), or to generate antibodies specific for CPA4 proteins. In a preferred embodiment, a CPA4 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to CPA4 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a CPA4 nucleic acid molecule of the invention is introduced, e.g., a CPA4 nucleic acid molecule within a recombinant expression vector or a CPA4 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a CPA4 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a CPA4 protein. Accordingly, the invention further provides methods for producing a CPA4 protein using the host cells of the invention. In one embodiment, the method comprises culturing the host cell of the invention (into which a recombinant expression vector encoding a CPA4 protein has been introduced) in a suitable medium such that a CPA4 protein is produced. In another embodiment, the method further comprises isolating a CPA4 protein from the medium or the host cell.

Isolated Nucleic Acid Molecules Used in the Methods of the Invention

The sequence of the isolated human CPA4 cDNA, the predicted amino acid sequence of the human CPA4 polypeptide, and the coding sequence comprising only the ORF are shown in SEQ ID NOs: 1, 2, and 3 respectively. The CPA4 sequences are also described in U.S. Pat. No. 5,747,336 and the PCT application WO 01/28995, the contents of which are incorporated herein by reference.

The methods of the invention include the use of isolated nucleic acid molecules that encode CPA4 proteins or biologically active portions thereof, as well as nucleic acid fragments sufficient for use as hybridization probes to identify CPA4-encoding nucleic acid molecules (e.g., CPA4 mRNA) and fragments for use as PCR primers for the amplification or mutation of CPA4 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, or a portion thereof, can be isolated using standard molecular biology techniques and the sequence information provided herein. Using all or portion of the nucleic acid sequence of SEQ ID NO:1 as a hybridization probe, CPA4 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO: 1 can be isolated by the polymerase chain reaction (PCR) using synthetic oligonucleotide primers designed based upon the sequence of SEQ ID NO:1.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to CPA4 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, a complement of the nucleotide sequence shown in SEQ ID NO:1, or a portion of any of these nucleotide sequences. A nucleic acid molecule which is complementary to the nucleotide sequence shown in SEQ ID NO:1, is one which is sufficiently complementary to the nucleotide sequence shown in SEQ ID NO:1 such that it can hybridize to the nucleotide sequence shown in SEQ ID NO:1 thereby forming a stable duplex.

In still another preferred embodiment, an isolated nucleic acid molecule used in the methods of the present invention comprises a nucleotide sequence which is at least about 70%, 75%, 80%, 85%, 90%, 95%, 96%, 97%, 98%, 99% or more identical to the entire length of the nucleotide sequence shown in SEQ ID NO:1 or a portion of any of this nucleotide sequence.

Moreover, the nucleic acid molecules used in the methods of the invention can comprise only a portion of the nucleic acid sequence of SEQ ID NO:1, for example, a fragment which can be used as a probe or primer or a fragment encoding a portion of a CPA4 protein, e.g., a biologically active portion of a CPA4 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 of an anti-sense sequence of SEQ ID NO:1 or of a naturally occurring allelic variant or mutant of SEQ ID NO: 1. In one embodiment, a nucleic acid molecule used in the methods of the present invention comprises a nucleotide sequence which is greater than 100, 100-200, 200-300, 300-400, 400-500, 500-600, or more nucleotides in length and hybridizes under stringent hybridization conditions to a nucleic acid molecule of SEQ ID NO: 1.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 4250° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na+] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a CPA4 protein, such as by measuring a level of a CPA4-encoding nucleic acid in a sample of cells from a subject e.g., detecting CPA4 mRNA levels or determining whether a genomic CPA4 gene has been mutated or deleted.

The methods of the invention further encompass the use of nucleic acid molecules that differ from the nucleotide sequence shown in SEQ ID NO:1 due to degeneracy of the genetic code and thus encode the same CPA4 proteins as those encoded by the nucleotide sequence shown in SEQ ID NO:1. In another embodiment, an isolated nucleic acid molecule included in the methods of the invention has a nucleotide sequence encoding a protein having an amino acid sequence shown in SEQ ID NO:2.

The methods of the invention further include the use of allelic variants of human and/or mouse CPA4, e.g., functional and non-functional allelic variants. Functional allelic variants are naturally occurring amino acid sequence variants of the human and/or mouse CPA4 protein that maintain a CPA4 activity. Functional allelic variants will typically contain only conservative substitution of one or more amino acids of SEQ ID NO:2, or substitution, deletion or insertion of non-critical residues in non-critical regions of the protein.

Non-functional allelic variants are naturally occurring amino acid sequence variants of the human and/or mouse CPA4 protein that do not have a CPA4 activity. Non-functional allelic variants will typically contain a non-conservative substitution, deletion, or insertion or premature truncation of the amino acid sequence of SEQ ID NO:2, or a substitution, insertion or deletion in critical residues or critical regions of the protein.

The methods of the present invention may further use non-human orthologues of the human and/or mouse CPA4 protein. Orthologues of the human and/or mouse CPA4 protein are proteins that are isolated from non-human organisms and possess the same CPA4 activity.

The methods of the present invention further include the use of nucleic acid molecules comprising the nucleotide sequence of SEQ ID NO:1 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of CPA4 (e.g., the sequence of SEQ ID NO:2) without altering the biological activity, whereas an “essential” amino acid residue is required for biological activity. For example, amino acid residues that are conserved among the CPA4 proteins of the present invention are not likely to be amenable to alteration.

Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a CPA4 protein is preferably replaced with another amino acid residue from the same side chain family. Alternatively, in another embodiment, mutations can be introduced randomly along all or part of a CPA4 coding sequence, such as by saturation mutagenesis, and the resultant mutants can be screened for CPA4 biological activity to identify mutants that retain activity. Following mutagenesis of SEQ ID NO:1 the encoded protein can be expressed recombinantly and the activity of the protein can be determined using the assay described herein.

Another aspect of the invention pertains to the use of isolated nucleic acid molecules which are antisense to the nucleotide sequence of SEQ ID NO: 1. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an anti-sense nucleic acid can hydrogen bond to a sense nucleic acid. The antisense nucleic acid can be complementary to an entire CPA4 coding strand, or to only a portion thereof. In one embodiment, an antisense nucleic acid molecule is antisense to a “coding region” of the coding strand of a nucleotide sequence encoding a CPA4. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding CPA4. The term “noncoding region” refers to 5′ and 3′ sequences which flank the coding region that are not translated into amino acids (also referred to as 5′ and 3′ untranslated regions).

Given the coding strand sequences encoding CPA4 disclosed herein, antisense nucleic acids of the invention can be designed according to the rules of Watson and Crick base pairing. The antisense nucleic acid molecule can be complementary to the entire coding region of CPA4 mRNA, but more preferably is an oligonucleotide which is antisense to only a portion of the coding or noncoding region of CPA4 mRNA. For example, the antisense oligonucleotide can be complementary to the region surrounding the translation start site of CPA4 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a CPA4 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave CPA4 mRNA transcripts to thereby inhibit translation of CPA4 mRNA. A ribozyme having specificity for a CPA4-encoding nucleic acid can be designed based upon the nucleotide sequence of a CPA4 cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a CPA4-encoding mRNA. See, e.g., Cech et al. U.S. Pat. No. 4,987,071; and Cech et al. U.S. Pat. No. 5,116,742. Alternatively, CPA4 mRNA can be used to select a catalytic RNA having a specific ribonuclease activity from a pool of RNA molecules. See, e.g., Bartel, D. and Szostak, J. W. (1993) Science 261:1411-1418.

Alternatively, CPA4 gene expression can be inhibited by targeting nucleotide sequences complementary to the regulatory region of the CPA4 (e.g., the CPA4 promoter and/or enhancers) to form triple helical structures that prevent transcription of the CPA4 gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioassays 14(12): 807-15.

In yet another embodiment, the CPA4 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-OKeefe et al. (1996) Proc. Natl. Acad. Sci. 93:14670-675.

PNAs of CPA4 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of CPA4 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-OKeefe et al. (1996) supra).

In another embodiment, PNAs of CPA4 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of CPA4 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Isolated CPA4 Proteins and Anti-CPA4 Antibodies Used in the Methods of the Invention

The methods of the invention include the use of isolated CPA4 proteins, and biologically active portions thereof, as well as polypeptide fragments suitable for use as immunogens to raise anti-CPA4 antibodies. In one embodiment, native CPA4 proteins can be isolated from cells or tissue sources by an appropriate purification scheme using standard protein purification techniques. In another embodiment, CPA4 proteins are produced by recombinant DNA techniques. Alternative to recombinant expression, a CPA4 protein or polypeptide can be synthesized chemically using standard peptide synthesis techniques.

As used herein, a “biologically active portion” of a CPA4 protein includes a fragment of a CPA4 protein having a CPA4 activity. Biologically active portions of a CPA4 protein include peptides comprising amino acid sequences sufficiently identical to or derived from the amino acid sequence of the CPA4 protein, e.g., the amino acid sequence shown in SEQ ID NO:2 or 5, which include fewer amino acids than the full length CPA4 proteins, and exhibit at least one activity of a CPA4 protein. Typically, biologically active portions comprise a domain or motif with at least one activity of the CPA4 protein (e.g., a region of the CPA4 protein that is believed to be involved in the regulation of metabolic activity). A biologically active portion of a CPA4 protein can be a polypeptide which is, for example, 25, 50, 75, 100, 125, 150, 175, 200, 250, 300 or more amino acids in length. Biologically active portions of a CPA4 protein can be used as targets for developing agents which modulate a CPA4 activity.

In a preferred embodiment, the CPA4 protein used in the methods of the invention has an amino acid sequence shown in SEQ ID NO:2. In other embodiments, the CPA4 protein is substantially identical to SEQ ID NO:2, and retains the functional activity of the protein of SEQ ID NO:2, yet differs in amino acid sequence due to natural allelic variation or mutagenesis, as described in detail in subsection V above. Accordingly, in another embodiment, the CPA4 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the CPA4 amino acid sequence of SEQ ID NO:2 having 361 amino acid residues, at least 108, preferably at least 144, more preferably at least 180, more preferably at least 217, even more preferably at least 253, and even more preferably at least 289 or 325 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use CPA4 chimeric or fusion proteins. As used herein, a CPA4 “chimeric protein” or “fusion protein” comprises a CPA4 polypeptide operatively linked to a non-CPA4 polypeptide. An “CPA4 polypeptide” refers to a polypeptide having an amino acid sequence corresponding to a CPA4 molecule, whereas a “non-CPA4 polypeptide” refers to a polypeptide having an amino acid sequence corresponding to a protein which is not substantially homologous to the CPA4 protein, e.g., a protein which is different from the CPA4 protein and which is derived from the same or a different organism. Within a CPA4 fusion protein the CPA4 polypeptide can correspond to all or a portion of a CPA4 protein. In a preferred embodiment, a CPA4 fusion protein comprises at least one biologically active portion of a CPA4 protein. In another preferred embodiment, a CPA4 fusion protein comprises at least two biologically active portions of a CPA4 protein. Within the fusion protein, the term “operatively linked” is intended to indicate that the CPA4 polypeptide and the non-CPA4 polypeptide are fused in-frame to each other. The non-CPA4 polypeptide can be fused to the N-terminus or C-terminus of the CPA4 polypeptide.

For example, in one embodiment, the fusion protein is a GST-CPA4 fusion protein in which the CPA4 sequences are fused to the C-terminus of the GST sequences. Such fusion proteins can facilitate the purification of recombinant CPA4.

In another embodiment, this fusion protein is a CPA4 protein containing a heterologous signal sequence at its N-terminus. In certain host cells (e.g., mammalian host cells), expression and/or secretion of CPA4 can be increased through use of a heterologous signal sequence.

The CPA4 fusion proteins used in the methods of the invention can be incorporated into pharmaceutical compositions and administered to a subject in vivo. The CPA4 fusion proteins can be used to affect the bioavailability of a CPA4 substrate. Use of CPA4 fusion proteins may be useful therapeutically for the treatment of disorders caused by, for example, (i) aberrant modification or mutation of a gene encoding a CPA4 protein; (ii) mis-regulation of the CPA4 gene; and (iii) aberrant post-translational modification of a CPA4 protein.

Moreover, the CPA4-fusion proteins used in the methods of the invention can be used as immunogens to produce anti-CPA4 antibodies in a subject, to purify CPA4 substrates and in screening assays to identify molecules which inhibit the interaction of CPA4 with a CPA4 substrate.

Preferably, a CPA4 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or staggerended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A CPA4-encoding nucleic acid can be cloned into such an expression vector such that the fusion moiety is linked in-frame to the CPA4 protein.

The present invention also pertains to the use of variants of the CPA4 proteins which function as either CPA4 agonists (mimetics) or as CPA4 antagonists. Variants of the CPA4 proteins can be generated by mutagenesis, e.g., discrete point mutation or truncation of a CPA4 protein. An agonist of the CPA4 proteins can retain substantially the same, or a subset, of the biological activities of the naturally occurring form of a CPA4 protein. An antagonist of a CPA4 protein can inhibit one or more of the activities of the naturally occurring form of the CPA4 protein by, for example, competitively modulating a CPA4 mediated activity of a CPA4 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the CPA4 protein.

In one embodiment, variants of a CPA4 protein which function as either CPA4 agonists (mimetics) or as CPA4 antagonists can be identified by screening combinatorial libraries of mutants, e.g., truncation mutants, of a CPA4 protein for CPA4 protein agonist or antagonist activity. In one embodiment, a variegated library of CPA4 variants is generated by combinatorial mutagenesis at the nucleic acid level and is encoded by a variegated gene library. A variegated library of CPA4 variants can be produced by, for example, enzymatically ligating a mixture of synthetic oligonucleotides into gene sequences such that a degenerate set of potential CPA4 sequences is expressible as individual polypeptides, or alternatively, as a set of larger fusion proteins (e.g., for phage display) containing the set of CPA4 sequences therein. There are a variety of methods which can be used to produce libraries of potential CPA4 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CPA4 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a CPA4 protein coding sequence can be used to generate a variegated population of CPA4 fragments for screening and subsequent selection of variants of a CPA4 protein. In one embodiment, a library of coding sequence fragments can be generated by treating a double stranded PCR fragment of a CPA4 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the CPA4 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CPA4 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify CPA4 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-CPA4 antibodies. An isolated CPA4 protein, or a portion or fragment thereof, can be used as an immunogen to generate antibodies that bind CPA4 using standard techniques for polyclonal and monoclonal antibody preparation. A full-length CPA4 protein can be used or, alternatively, antigenic peptide fragments of CPA4 can be used as immunogens. The antigenic peptide of CPA4 comprises at least 8 amino acid residues of the amino acid sequence shown in SEQ ID NO:2 and encompasses an epitope of CPA4 such that an antibody raised against the peptide forms a specific immune complex with the CPA4 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of CPA4 that are located on the surface of the protein, e.g., hydrophilic regions, as well as regions with high antigenicity.

A CPA4 immunogen is typically used to prepare antibodies by immunizing a suitable subject, (e.g., rabbit, goat, mouse, or other mammal) with the immunogen. An appropriate immunogenic preparation can contain, for example, recombinantly expressed CPA4 protein or a chemically synthesized CPA4 polypeptide. The preparation can further include an adjuvant, such as Freund's complete or incomplete adjuvant, or similar immunostimulatory agent. Immunization of a suitable subject with an immunogenic CPA4 preparation induces a polyclonal anti-CPA4 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a CPA4. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind CPA4 molecules. The term “monoclonal antibody” or “monoclonal antibody composition”, as used herein, refers to a population of antibody molecules that contain only one species of an antigen binding site capable of immunoreacting with a particular epitope of CPA4. A monoclonal antibody composition thus typically displays a single binding affinity for a particular CPA4 protein with which it immunoreacts.

Polyclonal anti-CPA4 antibodies can be prepared as described above by immunizing a suitable subject with a CPA4 immunogen. The anti-CPA4 antibody titer in the immunized subject can be monitored over time by standard techniques, such as with an enzyme linked immunosorbent assay (ELISA) using immobilized CPA4. If desired, the antibody molecules directed against CPA4 can be isolated from the mammal (e.g., from the blood) and further purified by well known techniques, such as protein A chromatography to obtain the IgG fraction. At an appropriate time after immunization, e.g., when the anti-CPA4 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a CPA4 immunogen as described above, and the culture supernatants of the resulting hybridoma cells are screened to identify a hybridoma producing a monoclonal antibody that binds CPA4.

Any of the many well known protocols used for fusing lymphocytes and immortalized cell lines can be applied for the purpose of generating an anti-CPA4 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from American Type Culture Collection (ATCC). Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind CPA4, e.g., using a standard ELISA assay.

Alternative to preparing monoclonal antibody-secreting hybridomas, a monoclonal anti-CPA4 antibody can be identified and isolated by screening a recombinant combinatorial immunoglobulin library (e.g., an antibody phage display library) with CPA4 to thereby isolate immunoglobulin library members that bind CPA4. Kits for generating and screening phage display libraries are commercially available (e.g., the Pharmacia Recombinant Phage Antibody System, Catalog No. 27-9400-01; and the Stratagene SurZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio-Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio-Technology 9:13731377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-CPA4 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:35213526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-CPA4 antibody can be used to detect CPA4 protein (e.g., in a cellular lysate or cell supernatant) in order to evaluate the abundance and pattern of expression of the CPA4 protein. Anti-CPA4 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as in the Sequence Listing are incorporated herein by reference.

### EXAMPLES

### Example 1

### CPA4 Gene Expression in Human and Mouse Tissues

Tissues were collected from 10 week old male C57/B16J mice purchased from Jackson Laboratories (Bar Harbor, Me.). Total RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control 18S gene, confirming efficient removal of genomic DNA contamination. CPA4 expression was measured by TaqMan quantitative PCR analysis, performed according to the manufacturer's directions (Perkin Elmer Applied Biosystems, Foster City, Calif.).

Tissue samples included the following normal human tissues: aorta, heart, veins, spinal cord, brain (cortex), glial cells, breast, ovary, pancreas, prostate, colon, kidney, liver, lung, spleen, tonsil, lymph node, thymus, skeletal muscle, skin, adipose, osteoblasts, osteoclasts, pancreatic islets, placenta, primary adipocytes, subcutaneous adipose adipocytes differentiated in vitro, preadipocytes, brain, and small intestine.

Normal mouse tissues examined included the following: brain/hypothalamus, hypothalamus, heart, lung, brown fat (BAT), white fat (WAT), liver, kidney, skeletal muscle, diaphysis, metaphysis, pancreas, spleen, colon, intestine, testis, osteoblast cell line ST-2, and Calvaria. PCR probes were designed by PrimerExpress software (PE Biosystems) based on the respective sequences of murine and human CPA4.

To standardize the results between the different tissues, two probes, distinguished by different fluorescent labels, were added to each sample. The differential labeling of the probe for the CPA4 and the probe for 18S RNA (as an internal control) thus enabled their simultaneous measurement in the same well. Forward and reverse primers and the probes for both 18S RNA and human or murine CPA4 were added to the TaqMan Universal PCR Master Mix (PE Applied Biosystems). Although the final concentration of primer and probe could vary, each was internally consistent within a given experiment. A typical experiment contained 200 nM each of the forward and reverse primers and 100 nM of the probe for the 18S RNA, as well as 600 nM of each of the forward and reverse primers and 200 nM of the probe for CPA4. TaqMan matrix experiments were carried out using an ABI PRISM 770 Sequence Detection System (PE Applied Biosystems). The thermal cycler conditions were as follows: hold for 2 minutes at 50° C. and 10 minutes at 95° C., followed by two-step PCR for 40 cycles of 95° C. for 15 seconds, followed by 60° C. for 1 minute.

The following method was used to quantitatively calculate CPA4 gene expression in the tissue samples, relative to the 18S RNA expression in the same tissue. The threshold values at which the PCR amplification started were determined using the manufacturer's software. PCR cycle number at threshold value was designated as CT. Relative expression was calculated as 2^{-((CTtest-CT18S) tissue of interest-(CTtest-CT18S) lowest expressing tissue in panel)}. Samples were run in duplicate and the averages of 2 relative expression levels that were linear to the amount of template cDNA with a slope similar to the slope for the internal control 18S were used.

The results of expression of CPA4 in human tissues by TaqMan analysis showed significant levels of expression of CPA4 in kidney, spinal cord, brain cortex and brain hypothalamic tissue. Furthermore, CPA4 was present in brain hypothalamic tissue at considerably higher levels compared to brain cortex (see e.g., Table 2). These data indicate that CPA4 is preferentially expressed in tissues relevant to regulation of metabolic disease, such as hypothalamus.

TaqMan analysis was also performed in mouse tissues as indicated above. Consistent with the human results, mCPA4 was highly expressed in brain and hypothalamus tissue, but was present at considerably lower levels in most other tissues tested (see e.g., Table 1). These data are in agreement with the human TaqMan expression data and demonstrate that the brain, particularly the hypothalamus, is a major site of CPA4 expression.

### Example 2

### Localization of CPA4 Expression

To further confirm the TaqMan expression data, additional Taqman analysis on tissues isolated from regions of mouse hypothalamus was assessed. Coronal sections from mouse brain 280 mm in thickness that span 3 mm caudal to the paraventricular nucleus and rostral to the arcuate nucleus (all within the hypothalamic region) were generated and stored at −80 C. Micro-dissection to isolate the body weight nuclei including the arcuate, paraventricular, dorsal medial, ventral medial areas as well as the lateral hypothalamic region were performed using Palvovitz micro-dissection needles (Stoelting Co.; Cat # 57401) for use in standard RNA extraction procedures and Taqman analysis as described above. Table 3 depicts relative expression level results, indicating CPA4 expression is highest in arcuate and paraventricular nuclei regions of the hypothalamus, and this expression is significantly higher than whole hypothalamus samples. These data are in agreement with the multi-panel mouse TaqMan expression data (Table 1) and show that CPA4 is highly expressed satiety control centers of the hypothalamus.

TABLE 1
m17683 Expression in
Mouse Multi-tissue Panel

| | Relative |
|---|---|
| Tissue Type | Expression |
| brain | 0.30 |
| hypothalamus | 5.11 |
| white fat | 0.09 |
| brown fat | 0.10 |
| intestine | 0.08 |
| colon | 0.10 |
| sk muscle | 0.13 |
| pancreas | 0.08 |
| calvaria | 0.09 |
| bone marrow | 0.06 |
| ovary | 0.08 |
| white fat (old) | 0.08 |
| liver | 0.07 |
| kidney | 0.08 |
| testis | 0.60 |
| lung | 0.09 |
| heart | 0.12 |
| spleen (old) | 0.08 |
| diaphysis | 0.11 |
| metaphysis | 0.06 |
| lung (old) | 0.31 |

TABLE 2
17683 Expression in Human Multi-Tissue Panel

| | Relative | | Relative |
|---|---|---|---|
| Tissue Type | Expression | Tissue Type | Expression |
| Aorta/Normal | 0.18 | Kidney/Normal | 0.45 |
| Fetal | 2.21 | Liver/Normal | 0.00 |
| Heart/Normal | | | |
| Heart/Normal | 0.03 | Liver/Fibrosis | 0.01 |
| Heart/CHF | 0.01 | Fetal Liver/Normal | 0.03 |
| Vein/Normal | 0.41 | Lung/Normal | 0.01 |
| SMC/Aortic | 0.90 | Lung/COPD | 0.26 |
| Nerve/Normal | 0.10 | Spleen/Normal | 0.02 |
| Spinal | 1.30 | Tonsil/Normal | 0.01 |
| Cord/Normal | | | |
| Brain | 5.00 | Lymph | 0.00 |
| Cortex/Normal | | Node/Normal | |
| Brain | 17.65 | Thymus/Normal | 1.38 |
| Hypothalmus/Normal | | | |
| Glial Cells | 0.1 | Epithelial Cells | 0 |
| (Astrocytes) | | (Prostate) | |
| Glioblastoma | 3.10 | Endothelial Cells | 1.98 |
| | | (Aortic) | |
| Breast/Normal | 0.02 | Skeletal | 0.19 |
| | | Muscle/Normal | |
| Breast/Tumor | 0.50 | Fibroblasts (Dermal) | 0.2 |
| Ovary/Normal | 0.76 | Adipose/Normal | 0.02 |
| Ovary/Tumor | 0.50 | Osteoblasts | 0.2 |
| | | (Primary) | |
| Pancreas/Normal | 0.02 | Osteoblasts (Undiff) | 0.7 |
| Prostate/Normal | 0.03 | Osteoblasts (Diff) | 3.16 |
| Prostate/Tumor | 0.01 | Osteoclasts | 0.00 |
| Colon/Normal | 0.00 | | |
| Colon/Tumor | 0.05 | | |
| Colon/IBD | 0.00 | | |
| Colon/Normal | 0.00 | | |

TABLE 3
Enriched CPA4 Expression in ARC/PVN

| | Relative CPA4 |
|---|---|
| Sample | Expression |
| ARC | 0.792 |
| LH | 0.234 |
| DMH | 0.332 |
| PVN | 1.001 |
| VMH | 0.123 |
| Hypothalamus | 0.108 |
| Brain | 0.029 |

### Example 3

### Regulation of CPA4 in Genetically Obese Mice

To determine whether CPA4 is regulated in obese, insulin-resistant mice, we examined expression in genetically obese ob/ob mice. CPA4 expression was measured as described above for samples isolated from ob/ob mice. CPA4 expression was enhanced in the hypothalamus of ob/ob mice as compared to wild-type control mice. In particular, expression in the arcuate nuclei of the hypothalamus demonstrated considerably higher levels of CPA4 expression as compared to wild type control mice (See Table 4).

TABLE 4
Enriched CPA4 Expression in Arcuate
Nuclei Hypothalamus of ob/ob mice

| Sample | Expression |
|---|---|
| WT ARC | 31.45 |
| WT Hypothalamus | 11.59 |
| WT brain/hypo | 3.33 |
| ob/ob ARC | 101.19 |
| ob/ob Hypothalamus | 26.84 |
| ob/ob brain/hypo | 1.00 |

### Example 4

### Overnight Fasting Conditions Increase Expression of CPA4

Expression regulation of CPA4 was also examined in response to a 16 hr overnight fasting conditions in the same manner as described above to determine relative changes in expression levels in response to changes in feeding conditions. Similar to expression of the orexigenic neuropeptides AGRP and NPY, CPA4 was induced in the arcuate nucleus after overnight fast (see Table 5).

TABLE 5
CPA4 expression is induced after overnight fast

| | | Increase in |
|---|---|---|
| | O/N Fast | Expression |
| AGRP | − | 1 |
| expression | + | 4.94 |
| NPY | − | 1 |
| expression | + | 4.58 |
| CPA4 | − | 1 |
| expression | + | 4.16 |

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method of identifying a nucleic acid molecule associated with a metabolic disorder comprising:
a) contacting a sample comprising nucleic acid molecules with at least one probe or primer comprising at least 25 contiguous nucleotides of SEQ ID NO: 1; and
b) detecting the presence of a nucleic acid molecule in the sample that hybridizes to the probe or is amplified under conditions that allow amplification, thereby identifying a nucleic acid molecule associated with a metabolic disorder.

2. The method of claim 1, wherein the hybridization probe is detectably labeled.

3. The method of claim 1, wherein the method is used to detect mRNA or genomic DNA in the sample.

4. A method of identifying a polypeptide associated with a metabolic disorder comprising:
a) contacting a sample comprising polypeptides with a CPA4 binding substance; and
b) detecting the presence of a polypeptide in the sample that binds to the CPA4 binding substance, thereby identifying a polypeptide associated with a metabolic disorder.

5. The method of claim 4, wherein the binding substance is an antibody.

6. The method of claim 4, wherein the binding substance is detectably labeled.

7. A method of identifying a subject having a metabolic disorder, or at risk for developing a metabolic disorder comprising:
a) contacting a sample obtained from the subject comprising nucleic acid molecules with at least one probe or primer comprising at least 25 contiguous nucleotides of SEQ ID NO:1; and
b) detecting the presence of a nucleic acid molecule in the sample that hybridizes to the probe or is amplified under conditions that allow amplification, thereby identifying a subject having a metabolic disorder, or at risk for developing a metabolic disorder.

8. The method of claim 7, wherein the hybridization probe is detectably labeled.

9. The method of claim 7, wherein the method is used to detect mRNA or genomic DNA in the sample.

10. A method of identifying a subject having a metabolic disorder, or at risk for developing a metabolic disorder comprising:
a) contacting a sample obtained from the subject comprising polypeptides with a CPA4 binding substance; and
b) detecting the presence of a polypeptide in the sample that binds to the CPA4 binding substance, thereby identifying a subject having a metabolic disorder, or at risk for developing a metabolic disorder.

11. The method of claim 10, wherein the binding substance is an antibody.

12. A method for identifying a compound capable of treating a metabolic disorder characterized by aberrant CPA4 nucleic acid expression comprising assaying the ability of the compound to modulate CPA4 nucleic acid expression, thereby identifying a compound capable of treating a metabolic disorder characterized by aberrant CPA4 nucleic acid expression.

13. The method of claim 12, wherein the metabolic disorder is a disorder associated with aberrant food intake.

14. The method of claim 13, wherein the disorder is selected from the group consisting of obesity, cachexia and anorexia.

15. A method for identifying a compound capable of treating a metabolic disorder characterized by aberrant CPA4 polypeptide activity comprising assaying the ability of the compound to modulate CPA4 polypeptide activity, thereby identifying a compound capable of treating a metabolic disorder characterized by aberrant CPA4 polypeptide activity.

16. The method of claim 15, wherein the metabolic disorder is a disorder associated with aberrant food intake.

17. The method of claim 15, wherein the disorder is selected from the group consisting of obesity, cachexia and anorexia.

18. The method of claim 15, wherein the ability of the compound to modulate the activity of the CPA4 polypeptide is determined by detecting carboxypeptidase activity.

19. The method of claim 15, wherein the ability of the compound to modulate the activity of the CPA4 polypeptide is determined by identifying a compound which binds the CPA4 polypeptide.

20. A method for treating a subject having a metabolic disorder characterized by aberrant CPA4 polypeptide activity or aberrant CPA4 nucleic acid expression comprising administering to the subject a CPA4 modulator, thereby treating the subject having a metabolic disorder.
